(19) United States
(12) Patent Application Publication
(10) Pub. No.: US 2013/0309660 A1
(43) Pub. Date: Nov. 21, 2013

(54) METHODS OF CHARACTERIZING, DETERMINING SIMILARITY, PREDICTING CORRELATION BETWEEN AND REPRESENTING SEQUENCES AND SYSTEMS AND INDICATORS THEREFOR

(71) Applicant: Real Time Genomics, Inc., (US)

(72) Inventors: Stuart John INGLIS, Hamilton (NZ); Leonard Eric TRIGG, Ngahinapouri (NZ); John Gerald CLEARY, Hamilton (NZ); Sean Alistair IRVINE, Hamilton (NZ); Richard Henry LITTIN, San Francisco, CA (US); Leonard Nathan BLOKSBERG, Manukau (NZ)

(73) Assignee: Real Time Genomics, Inc., San Bruno, CA (US)

(21) Appl. No.: 13/848,653

(22) Filed: Mar. 21, 2013

Related U.S. Application Data
(63) Continuation of application No. PCT/NZ2011/000197, filed on Sep. 23, 2011.

(30) Foreign Application Priority Data
Sep. 23, 2010 (NZ) 588163

(51) Int. Cl. C12Q 1/68; G06F 19/22

(52) U.S. Cl. CPC C12Q 1/6869; G06F 19/22
USPC 435/6.1; 435/287.2; 702/20

(57) ABSTRACT

A computer implemented method for characterizing one or more sequences by generating index values representing portions of the sequences and finding characterizing index values based on a comparison of the index values. The index values may be obtained by applying one or more mask over each sequence. The modified masks may have associated weightings and index values obtained using modified masks may be retained in the index only if the weightings are above a threshold value. Characterising index values may also be assessed for for their degree of uniqueness. Characterizing indexes may be used for predicting correlation between a sample sequence and one or more reference sequences. Biological monitoring systems utilising the characterizing index values are also disclosed. A biological indicator may be generatgenerated using one or more characterizing index values obtained by the above method and be used to produce an indicator that undergoes a property change in the presence of the one or more sequence.

### CROSS-REFERENCE TO RELATED APPLICATIONS

This application is a continuation of PCT International Patent Application No. PCT/NZ2011/000197, filed Sep. 23, 2011, which claims priority to New Zealand Patent Application No. 588163, filed Sep. 23, 2010, both of which are incorporated herein by reference in their entirety.

### FIELD OF THE INVENTION

The present invention relates to methods for characterizing a sequence, determining the similarity between sequences, predicting correlation between sequences based on characterizing index values and methods of graphically representing such information. The invention also relates to monitoring systems and indicators for detecting the presence of target sequences.

### BACKGROUND TO THE INVENTION

In nature there are numerous patterns that can be interpreted as sequences of discrete units. In biology, the sequence of nucleotides in DNA or RNA, and the sequences of amino acids in proteins are of particular interest. In DNA, sequences consist of discrete units which may take on one of the values A, C, G, T, while in RNA sequences, the values are A, C, G, and U. Proteins represent a more complicated sequence, as individual units may be one of 21 or more amino acids—in general 22 amino acids.

Sequencing machines are used to produce a machine readable encoding of such biological sequences. These machines use a variety of techniques to interpret the molecular information, and may introduce errors into the data in both systematic and random ways. Errors can usually be categorised into substitution errors, where the real code is substituted with an incorrect code (for example A swapping with G in DNA), or so called indel errors (insertion/deletion), where a random unit is inserted (for example AGT becoming AGCT in DNA) or deleted (for example AGTA becoming ATA).

A sequencing machine may produce a number of ‘reads’, where each read is a small length of coding a section of a genome sequence sample molecule, for example a 3 billion long DNA collection of chromosomes may have reads of only 100 units in length. Due to the method of generating the reads, the original position of each read against the original sequence is unknown, and so aligning techniques must be used to determine the original location of the reads. Typically alignment will need to take into account that the direction of the reads is also unknown.

Sequences or collections of sequences may be broken down into index values which may be hashed and used as an index to record the occurrence of index values in sequences. The applicant's prior application PCT/NZ2009/000245 published as WO2010/056131, the disclosure of which is incorporated herein by reference, describes methods of forming such indexes using masking techniques and using such indexes for sequence alignment.

There is a need to identify and/or explore characterizing similarities between sequences and to date this is often performed manually, which is time consuming and slow.

It is known to use “wet” or chemical processes to extract candidate genes (using gene expression measures or similar) that may be different in two species (or species groups). The genes in a specific set (for example, a virulent strain) may be different from a non-virulent set of strains. One of the genes that cause the virulent nature may be identified, the gene may then be sequenced and a characterizing sequence is extracted. Such approaches do not encompass the entire (or at least a large portion of) sequence information of an organism to obtain a number of characterizing sequences and select preferred characterising sequences.

There is also a need for efficient methods, systems and indicators to detect correlation between sequences such as in environmental monitoring applications.

It is an object of the invention to provide methods, systems and indicators meeting these needs or to at least provide the public with a useful choice.

### SUMMARY OF THE INVENTION

According to a first aspect there is provided a computer implemented method for characterizing one or more selected sequences, including the steps of:
a. generating index values representing portions of a plurality of sequences; and
b. determining characterizing index values for the one or more selected sequences of the plurality of sequences based on a comparison of the index values.

The characterizing index values may include all index values of the sequences. The index values may be obtained by applying one or more mask over each sequence. At least some of the masks may be modified masks that introduce sequence modifications. Some types of modifications may include changes, sequence insertions, sequence deletions and sequence repositioning. One index may contain the results using an unmodified mask (i.e. simple sliding window) and one or more further indexes may be created using modified masks. The modified masks may have associated weightings and index values obtained using modified masks may be retained in the index only if the weightings are above a threshold value.

Alternatively only common index values for the sequences may be retained. The sequences may be from a common family.

Alternatively only index values unique to the selected sequences may be retained. Index values of the selected sequences may be compared with index values of the other sequences to speed up identification of the unique index values.

Characterising index values may also be assessed for for their degree of uniqueness (i.e. whilst a cat is unique amounst dogs it is far more unique amoungst ladybirds). Such uniqueness may be assessed in terms of “sequential differentiation” (i.e. an index differing from all others by four bases is more unique that one differeing by one) and “contextual differentiation” (i.e. due to the chemistry or some other factor a particular sequence may be rare and so have particular differentiation)

Alternatively index values may be retained based on one or more rules. Index values may be retained for a plurality of selected sequences if the index value is unique to a number of selected sequences above a threshold value (e.g. more than 90%). Alternatively or additionally index values may be retained for a plurality of selected sequences if the characterizing index values are unique to the selected sequences and each selected sequence includes at least one characterizing index value.

There is further provided a computer implemented method for predicting correlation between a sample sequence and one or more reference sequences, including the steps of:
a. obtaining a characterizing index from the set of reference sequences by the method of any one of claims 1 to 13;
b. creating an index from the sample sequence;
c. comparing the sample sequence index with the characterizing index; and
d. identifying if there is a correlation between the sample sequence index and the characterizing index.

There is also provided a method for identifying target biological sequences including the steps of:
a. receiving a biological sample;
b. sequencing the biological sample to produce sequences of the genetic material of the biological sample;
c. creating an index of the biological sample sequences; and
d. detecting the presence target biological sequences by comparing the obtained index of biological sample sequences with an index of characterizing index values obtained as per one of the above methods.

A positive detection may require a comparison threshold to be exceeded. The comparison threshold may require the number of obtained index values matching the characterizing index values to exceed a threshold value. The characterizing index values may be weighted and the comparison threshold may require the cumulative weightings of matching index values to exceed a threshold value. The relative uniqueness of the characterising index values may also be taken into account.

There is further provided a method of producing a biological indicator by generating one or more characterizing index values by the above method and producing an indicator that undergoes a property change in the presence of the one or more sequence. Multiple characterizing index values may be aligned to form a longer more unique characterizing index value.

The property may be a visual property of the indicator, such as size, colour, luminescence etc. The indicator may be a string of enzymes that activate an element associated with the string of enzymes when in the presence of the one or more sequence.

There is further provided a biological monitoring system for identifying target biological sequences including:
a. a biological sample acquisition device;
b. a sequencer for sequencing the biological sample to produce sequences of genetic material of the biological sample;
c. memory storing an index of one or more index values characteristic of one or more target biological sequences; and
d. a processor for creating an index of the biological sample sequences and comparing the obtained index of biological sample sequences with one or more characteristic index of one or more target biological sequences and outputting an indication of correlation.

The characterizing index may be produced according to the method above. The index may include modified index values derived using masks that modify sequence values. Weightings may be associated with modified index values and correlation may be indicated when the cumulative weightings for matching index values exceeds a threshold.

There is also provided a method for determining a level of similarity between one or more first sequences and one or more second sequences, including the steps of:
a. generating one or more second index values representing portions of each second sequence;
b. providing one or more masks, wherein each mask has an associated weighting value based on the differences introduced by the mask;
c. for each mask, generating one or more first index values representing portions of the first sequence, wherein the mask is used to modify each portion of the first sequence before generating the corresponding first index values;
d. for each second sequence, calculating a score based at least in part on the number of first index values equal to a second index value of a second sequence weighted by a weighting value associated with the mask; and
e. producing a total score indicating the level of similarity based on the scores for each first index value.

Weighting may be based on one or more of: the type of sequence, chemistry of the sequences, sequence equipment characteristics and user specified criteria. Scoring may be modified based on feedback in relation to past scoring. This may be based on user feedback or automated analysis as to the quality of scoring based on performance metrics. The score associated with any index value may be related to the level of uniqueness of the value.

There is further provided a method of graphically representing a plurality of sequences including the steps of:
a. determining similarity between the sequences according to one of the above methods;
b. associating sequences based on their similarity;
c. graphically representing the sequences based on their similarity.

The graphical representation may be a tree, such as a tree of life, and selecting a branch of the tree may cause the branch to be separately represented.

There is also provided a method of graphically representing a plurality of sample sequences comprising:
a. determining the correlation between one or more sample sequences and a plurality of reference sequences;
b. performing dimension reduction on the correlation results; and
c. displaying the dimensionally reduced correlation results.

The reference sequences may be the characterizing index values obtained by the method above. The correlation results may be normalised before dimension reduction by subtracting the mean correlation result value and scaling the correlation results.

The dimension reduction may be principal component analysis, such as a dot plot, or singular value decomposition etc. The results for each sample have a different optical characteristic such as different colours.

A user may control correlation parameters, such as the length of each reference sequence, the sampling rate for each reference sequence, the dimensions to be reduced to observe the impact on a visual representation. The graphical representations for different correlation parameters are presented as an animation.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The accompanying drawings which are incorporated in and constitute part of the specification, illustrate embodiments of the invention and, together with the general description of the invention given above, and the detailed description of embodiments given below, serve to explain the principles of the invention.

FIG. 1 shows schematically a method of forming a characterizing index;

FIGS. 2a to 2d show possible rules employed to form a characterizing index;

FIG. 3 shows a continuous monitoring system;

FIG. 4 shows a sequencing machine contamination detection system;

FIG. 5 shows a phylogenetic, or tree of life, representation of a set of sequences;

FIG. 6 shows a branched representation of a set of sequences; and

FIG. 7 shows a representation of a comparison between two genomes after representation reduction.

### DETAILED DESCRIPTION

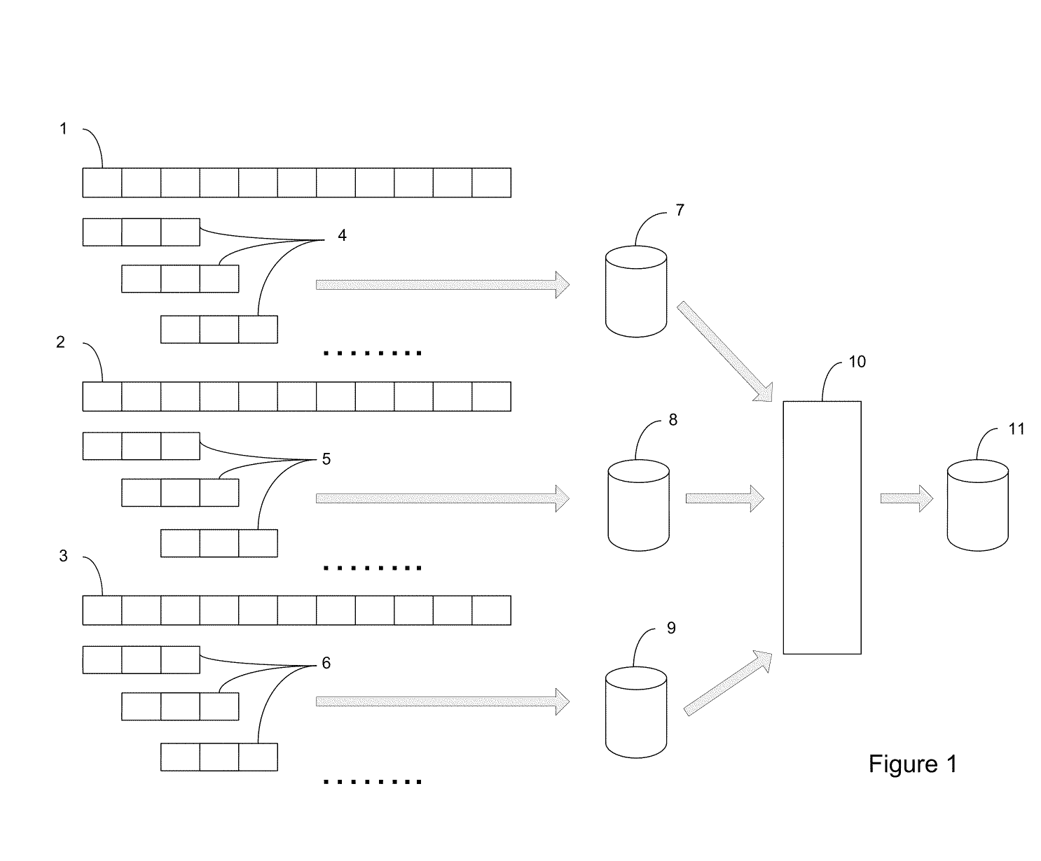

Referring to FIG. 1 a schematic diagram shows a method for forming a database of characterizing index value(s). In this example sequences 1, 2 and 3 have a simple sliding window applied to them to produce index values 4, 5 and 6. It is to be appreciated that this is a pictorial illustration to assist understanding and does not necessary reflect any physical implementation. It will be appreciated that there may be any desired number of sequences and associated databases.

The indexes 4, 5 and 6 for each sequence 1, 2 and 3 are stored in index databases 7, 8 and 9 in this example (there could be more where multiple masks are applied). The indexes from databases 7, 8 and 9 are supplied to a rules engine 10 which processes the indexes to produce a characterizing database 11.

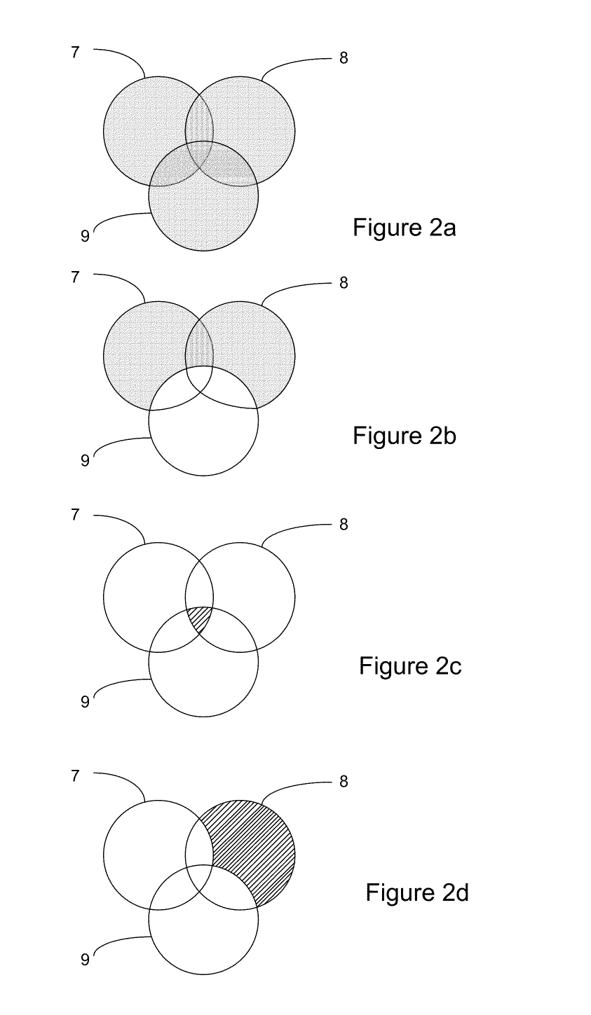

FIGS. 2a to 2d illustrate using Venn diagrams some simple logical operations that may be applied by rules engine 10 to illustrate the method. In the example shown FIG. 2a all index values for all sequences are stored in characterizing database 11. This may be useful where it is desired to know whether an index value for a sample could be from a sequence of the family of the sequences 1 to 3.

In FIG. 2b it is desired to form a characterizing database with index values from sequences 1 and 2 but not sequence 3. In order to accelerate processing the index values of the selected sequences 1 and 2 may be identified first and compared with index values of the other sequences to speed up identification of the unique index values.

In FIG. 2c it is desired to form a characterizing database containing only index values common to all sequences.

In FIG. 2d it is desired to form a characterizing database containing only index values unique to sequence 2. This is useful where it is desired to use detection of a single index value to indicate the presence of a sequence such as in biological monitoring applications. A rule such as shown in FIG. 2b may be used where it is desired to select one of a number of sequences of a given family of sequences.

The above description describes a simple rules engine in an ideal situation where perfect reference sequences and sample sequences are compared. In the real world there will typically be insertions, deletions and substitutions to deal with.

It will be appreciated that masks having insertions, deletions and substitutions may also be applied to the sequences 1, 2 and 3 to produce additional index values as disclosed in PCT/NZ2009/000245. Index values may have an associated weighting based on the modification introduced by a mask. Masks with multiple modifications or less likely modifications may be weighted accordingly. Alternatively index values for each mask may be stored in an associated database having an associated weighting. The weightings for a particular index value may be accumulated and the index only retained if it has a total weighting above a threshold.

Index values may be retained based on one or more threshold rules. Index values may be retained for a plurality of selected sequences if the index value is unique to a number of selected sequences above a threshold value (e.g. more than 80% of selected sequences). Alternatively or additionally index values may be retained for a plurality of selected sequences if the characterizing index values are unique to the selected sequences and each selected sequence includes at least one characterizing index value (i.e. there is coverage of unique index values across all selected sequences).

Characterising index values may also be assessed for for their degree of uniqueness (i.e. whilst a cat is unique amounst dogs it is far more unique amoungst ladybirds). Such uniqueness may be assessed in terms of “sequential differentiation” (i.e. an index differing from all others by four bases is more unique that one differeing by one) and “contextual differentiation” (i.e. due to the chemistry or some other factor a particular sequence may be rare and so have particular differentiation). This degree of uniqueness may be used in characterising index selection and as a weighting factor in evaluation of sample sequences.

Characterizing index values may also be aligned and combined to form longer and more unique characterizing indexes.

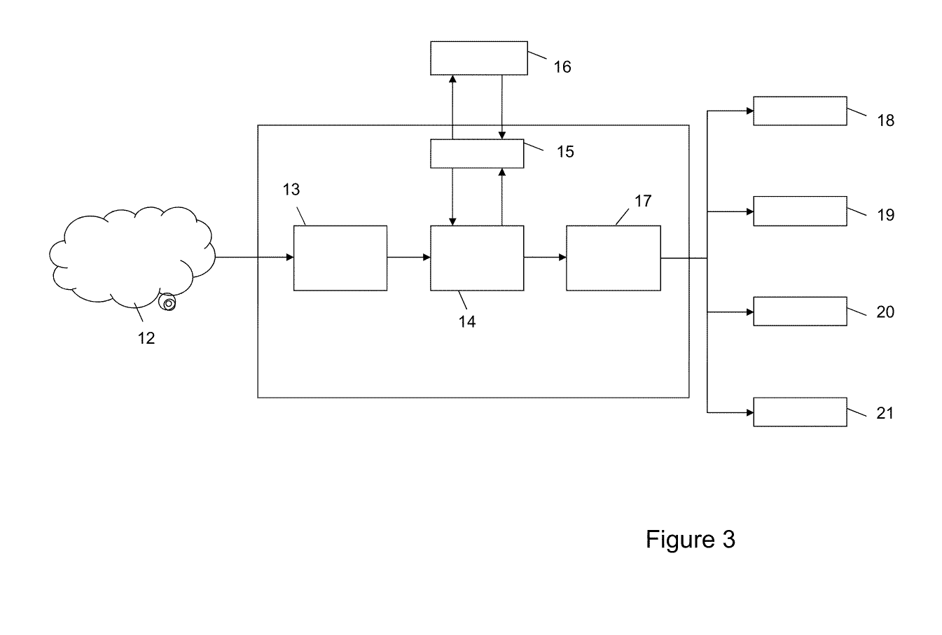

Referring to FIG. 3 there is shown a continuous monitoring system in which a biological sample 12 is sequenced by a sequencer 13 and the sample data sequence is supplied to data processing unit 14. Memory 15 stores characterizing indexes produced as described above, which may be updated over network 16. Data processing unit 14 generates indexes using a sliding window and compares these to characterizing indexes stored in memory 15. Depending upon the application and desired sensitivity different criteria may be set for different outputs. A single match between a sample index value and a characterizing index value may be enough to trigger an alarm. In other applications a threshold may have to be exceeded (e.g. a number of index matches must be exceeded). Different outputs may be triggered at different levels so that an entry may be written to a data record 19 for each match whereas a higher threshold may be required to trigger an alarm 18. Likewise information may be displayed on display 20 or sent to a remote unit 21 for predefined thresholds.

Where masked values are employed they may have associated weightings based on the likely reliability of a match based on the index. Weighting may be based on one or more of: the type of sequence, chemistry of the sequences, sequence equipment characteristics and user specified criteria. The weightings may also be based on statistical information as to the reliability of an index in indicating the presence of a target sequence. The cumulative value of weightings for matching index values may need to exceed a given threshold to activate an alarm in such situations.

Scoring may be modified based on feedback in relation to past scoring. This may be based on user feedback or automated analysis as to the quality of scoring based on performance metrics. The score associated with any index value may be related to the level of uniqueness of the value.

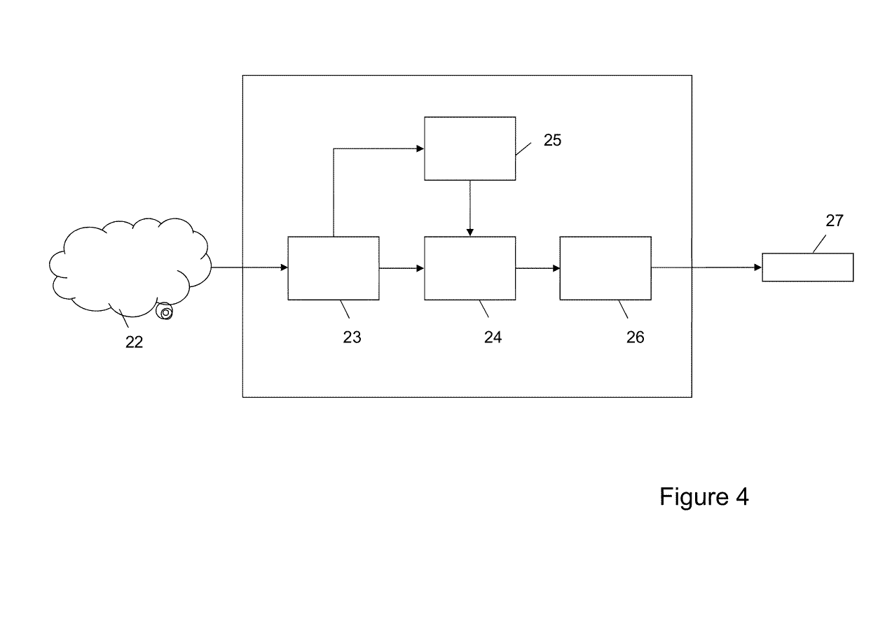

FIG. 4 shows a sequencing machine including a contamination detector using reference characterizing index values produced by a method described above. Sequencer 23 may sequence biological samples and provide data sequences to data processor 24. Contamination detection unit 25 may receive the data sequences too and generate index values using a sliding window and compare them to characterizing index values stored in memory obtained by a method described above. As described in relation to the monitoring apparatus a threshold of a single match or some more complex threshold may be set for contamination unit 25 to signal to data processor that contamination has occurred. Output unit 26 may signal contamination to user interface 27 and sequencing may be terminated.

The characterizing index values may also be used to produce biological indicators by producing an indicator that undergoes a property change in the presence of the one or more sequence. The property may be a visual property of the indicator, such as colour, luminescence etc. The indicator may be a string of enzymes that activate an element associated with the string of enzymes when in the presence of the one or more sequence.

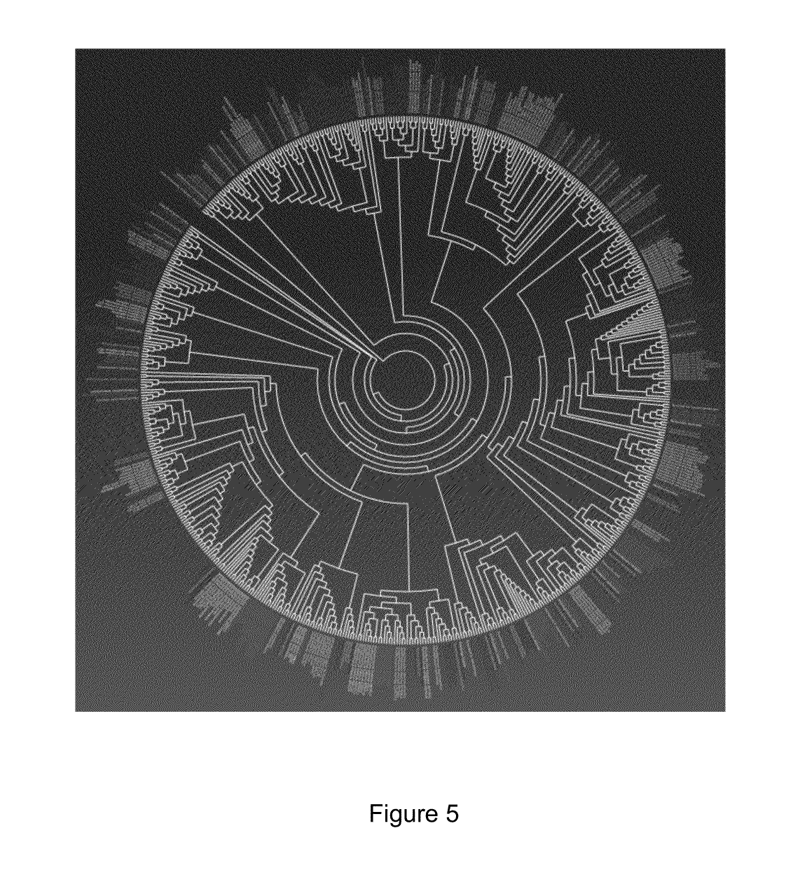

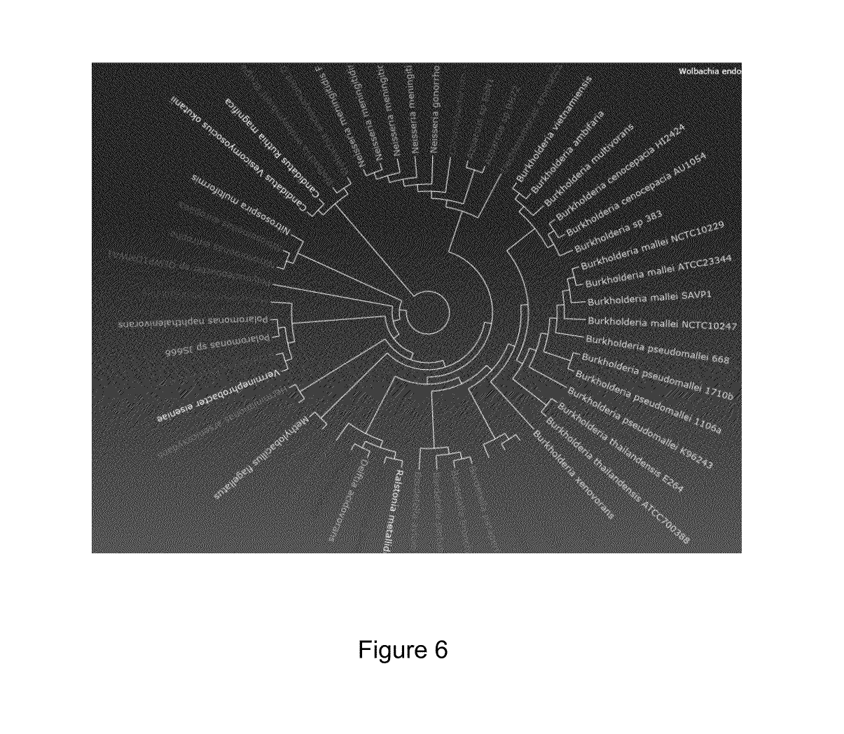

The characterizing index values may also be used to visually represent a set of sequences. For example indexes may be created for a family of sequences and common index values may be used to associate indexes in a visual representation. As shown in FIG. 5 a family of indexes may be represented in a “tree of life” representation. To assist exploration of the sequences the tree may be rotated using an input device and clicking on the branch of a tree may display a selected sub-branch in its own “tree of life” as shown in FIG. 6. Individual sequences may be selected in the tree of life representation and analysed using additional tools.

An alternative method of graphically representing a plurality of sample sequences involves the steps of:
a. determining the correlation between one or more sample sequences and a plurality of reference sequences (esp. characterizing index values);
b. performing dimension reduction on the correlation results; and
c. displaying the dimensionally reduced correlation results.

Dimension reduction techniques enable a coordinate system to be selected to best illustrate variance. This technique is particularly well suited to utilise human visual capabilities to assess results. The correlation results may be normalised before dimension reduction by subtracting the mean correlation result value and scaling the correlation results.

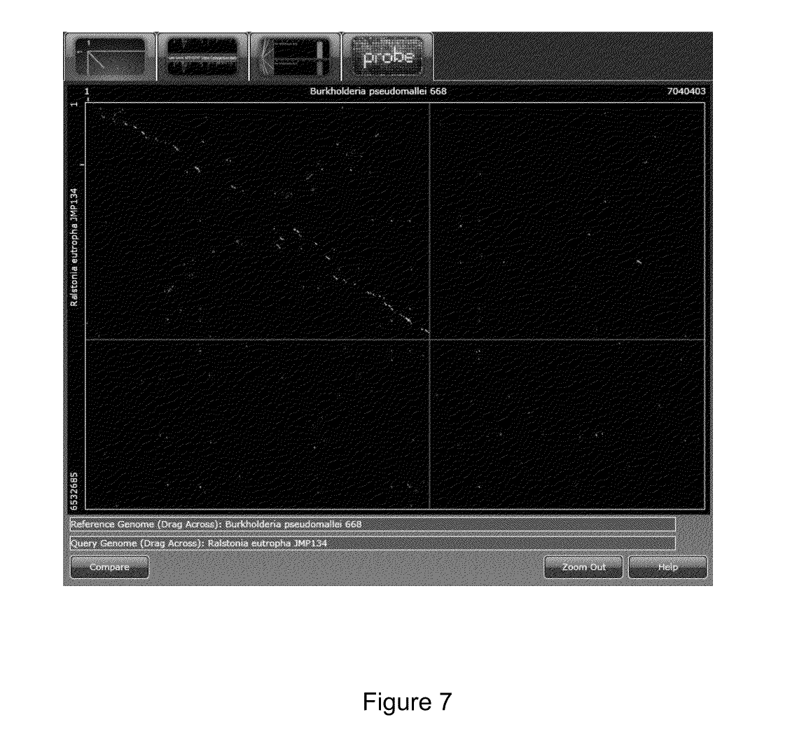

The dimension reduction may use principal component analysis techniques, such as a dot plot, or singular value decomposition etc. The results for each sample may be given a different optical characteristic such as different colour. This enables a user to easily see characteristics of each sample and relationships with other samples. A sample representation of the comparison of two sequences after dimension reduction is shown in FIG. 7.

Changes in correlation parameters may produce revealing results. For example if a small change in a parameter produces a large change in observed correlation in a dot plot then the strength of the correlation may be questioned. On the other hand consistency of correlation results with changing correlation parameters may give confidence in correlation results.

A user may control correlation parameters, such as the length of each reference sequence (i.e. number of bases per index value), the sampling rate for each reference sequence (e.g. one index value of length 16 bases per 25 bases), the dimensions to be reduced etc. to observe the impact on a visual representation. The graphical representations for different correlation parameters are presented as an animation. This enables the variance in correlation results of dot plots with different correlation parameters to be easily observed.

To avoid clutter of representations contiguous or overlapping index values may be consolidated into larger index values. The longer index values may be ascribed a higher confidence level.

The present invention thus provides methods for producing characterizing index values to simplify detection of target sequences and to facilitate investigation and research into sequences. Monitoring apparatus using the characterizing indexes can be less complex that traditional devices and significantly reduce processing time so as to be capable of performing real time biological monitoring. There is also provided a sequencing machine including on the fly monitoring of samples to detect contaminants and avoid lengthy processing of contaminated samples. There are also provided tools facilitating research into groups of sequences.

The invention sequences as much of the organism as possible and uses the underlying sequence data as input to the data analysis stages. The invention allows characterizing sequences to be formed without bias, as characterizing sequences may occur inside gene regions, but may also occur in non-coding regions. This invention uses all information and does not bias the determination of the characterizing sequences by a-priori knowledge.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and methods, and illustrative examples shown and described. Accordingly, departures may be made from such details without departure from the spirit or scope of the Applicant's general inventive concept.

1. A computer implemented method for characterizing one or more selected biological sequences, comprising:
a. generating index values representing portions of a plurality of biological sequences; and
b. determining characterizing index values for the one or more selected sequences of the plurality of sequences based on a comparison of the index values.

2. The method as claimed in claim 1 wherein the index values include all values of the sequences.

3. The method as claimed in claim 2 wherein the index values are obtained by applying one or more masks over each sequence.

4. The method as claimed in claim 3 wherein a plurality of masks are applied to each sequence and at least some of the masks are modified masks that introduce sequence modifications.

5. The method as claimed in claim 4 wherein a first index is created using an unmodified mask and one or more further indexes are created using modified masks.

6. The method as claimed in claim 4 wherein modified masks have associated weightings and index values obtained using modified masks are retained in the index only if the weightings are above a threshold value.

7. The method as claimed in claim 1 wherein common index values for the sequences are retained.

8. The method as claimed in claim 7 wherein the sequences are from a common family.

9. The method as claimed in claim 1 wherein only index values unique to the selected sequences are retained.

10. The method as claimed in claim 9 wherein only the index values of the selected sequences are compared with index values of the other sequences.

11. The method as claimed in claim 1 wherein index values are retained based on one or more rules.

12. The method as claimed in claim 11 wherein index values are retained for a plurality of selected sequences if the index value is unique to a number of selected sequences above a threshold value.

13. The method as claimed in claim 11 wherein index values are retained for a plurality of selected sequences if the characterizing index values are unique to the selected sequences and each selected sequence includes at least one characterizing index value.

14. A computer implemented method for predicting correlation between a sample biological sequence and one or more reference sequences, comprising:
a. obtaining a characterizing index from the set of reference sequences by the method of claim 1;
b. creating an index from the sample sequence;
c. comparing the sample sequence index with the characterizing index; and
d. identifying if there is a correlation between the sample sequence index and the characterizing index.

15. A method for identifying target biological sequences comprising:
a. receiving a biological sample;
b. sequencing the biological sample to produce sequences of the genetic material of the biological sample;
c. creating an index of the biological sample sequences; and
d. detecting the presence of target biological sequences by comparing the obtained index of biological sample sequences with an index of characterizing index values obtained as per the method of claim 1.

16. The method as claimed in claim 15 wherein a positive detection requires a comparison threshold to be exceeded.

17. The method as claimed in claim 16 wherein the comparison threshold requires the number of obtained index values matching the characterizing index values to exceed a threshold value.

18. The method as claimed in claim 16 wherein the characterizing index values are weighted and the comparison threshold requires the cumulative weightings of matching index values to exceed a threshold value.

19. A method of producing a biological indicator by generating one or more characterizing index values for one or more selected sequences by the method of claim 1 and producing an indicator that undergoes a property change in the presence of the one or more sequences.

20. The method as claimed in claim 19 wherein the property is a visual property of the indicator.

21.-22. (canceled)

23. The method as claimed in claim 20 wherein the indicator is a string of enzymes that activate an element associated with the string of enzymes when in the presence of the one or more sequences.

24. A biological monitoring system for identifying target biological sequences comprising:
a. a biological sample acquisition device;
b. a sequencer for sequencing a biological sample to produce sequences of genetic material of the biological sample;
c. memory storing an index of one or more index values characteristic of one or more target biological sequences; and
d. a processor capable of creating an index of the biological sample sequences and comparing the obtained index of biological sample sequences with one or more characteristic index of one or more target biological sequences and outputting an indication of correlation.

25. The system as claimed in claim 24 wherein the characterizing index is produced by:
a. generating index values representing portions of a plurality of biological sequences; and
b. determining characterizing index values for one or more selected sequences of the plurality of sequences based on a comparison of the index values.

26. The system as claimed in claim 24 wherein the index comprises modified index values derived using masks that modify sequence values.

27. The system as claimed in claim 26 wherein weightings are associated with modified index values and correlation is indicated when the cumulative weightings for matching index values exceeds a threshold.

28. A method for determining a level of similarity between one or more first sequences and one or more second sequences, comprising:
a. generating one or more second index values representing portions of each second sequence;
b. providing one or more masks, wherein each mask has an associated weighting value based on differences introduced by the mask;
c. for each mask, generating one or more first index values representing portions of the first sequence, wherein the mask is used to modify each portion of the first sequence before generating the corresponding first index values;
d. for each second sequence, calculating a score based at least in part on the number of first index values equal to a second index value of a second sequence weighted by a weighting value associated with the mask; and
e. producing a total score indicating the level of similarity based on the scores for each first index value.

29. The method as claimed in claim 28 wherein weighting is based on one or more of: the type of sequence, chemistry of the sequences, sequence equipment characteristics and user specified criteria.

30. The method as claimed in claim 28 wherein scoring is modified based on feedback in relation to past scoring.

31. The method as claimed in claim 28 wherein the score associated with any index value is related to the level of uniqueness of the value.

32.-50. (canceled)

51. The method as claimed in claim 1 wherein characterizing index values are selected at least in part on the basis of uniqueness.

52. The method as claimed in claim 51 wherein uniqueness is determined at least in part based on sequential differentiation or contextual differentiation.

53. (canceled)

54. The method as claimed in claim 1 wherein characterizing index values are aligned and combined to form longer characterizing index values.

55. The method as claimed in claim 51 wherein uniqueness is determined at least in part based on sequential differentiation.

56. The method as claimed in claim 20 wherein the visual property is color.

57. The method as claimed in claim 20 wherein the visual property is luminescence.
